(19) United States
(12) Patent Application Publication
(10) Pub. No.: US 2022/0175686 A1
(43) Pub. Date: Jun. 9, 2022

(54) NANOPARTICLE, METHOD FOR PRODUCING NANOPARTICLE, AND PHARMACEUTICAL COMPOSITION

(71) Applicants: Tadahiko Morinaga, Kanagawa (JP); Tatsuru Moritani, Kanagawa (JP); Yuichi Sato, Kanagawa (JP)

(72) Inventors: Tadahiko Morinaga, Kanagawa (JP); Tatsuru Moritani, Kanagawa (JP); Yuichi Sato, Kanagawa (JP)

(73) Assignee: Ricoh Company, Ltd., Tokyo (JP)

(21) Appl. No.: 17/439,911

(22) PCT Filed: Mar. 18, 2020
(86) PCT No.: PCT/JP2020/012145

§ 371 Date: Sep. 16, 2021

(30) Foreign Application Priority Data
Mar. 20, 2019 (JP) 2019-052206

(51) Int. Cl. A61K 9/51; A61K 38/13; A61K 31/5377; A61K 31/196; B01J 2/06; B01J 2/18

(52) U.S. Cl. CPC A61K 9/5123; A61K 9/5192; A61K 38/13; B01J 2/18; A61K 31/196; B01J 2/06; A61K 31/5377

(57) ABSTRACT

A nanoparticle includes a poorly-water-soluble physiologically active compound and an additive substance. A relative span factor (R.S.F) of the nanoparticle satisfies formula: 0<(R.S.F)≤1.0, a volume average particle diameter of the nanoparticle is 200 nm or less, and the poorly-water-soluble physiologically active compound is covered with the additive substance.

### TECHNICAL FIELD

The present disclosure relates to a nanoparticle, a method for producing a nanoparticle, and a pharmaceutical composition.

### BACKGROUND ART

In recent years, researches related to a drug delivery system have been actively carried out as technologies for administrating a medical component efficiently and safely to a disease site. Among such technologies, high in demand is a technology for forming a medical component into nanoparticles having particle diameters of several hundred nanometers or smaller is increased in order to deliver the medical component into blood vessels.

Generally, a sterilization treatment is often desired to perform on pharmaceuticals. There are a number of sterilization treatment methods. Since a filtering sterilization treatment using a filter having an opening size of 0.22 micrometers is simple, it is desired that a particle diameter of a nanoparticle be set to 200 nm or smaller.

Recently, moreover, researches on polypeptide or a kinase inhibitor that is a molecular target drug have been actively performed.

For example, proposed is a method for producing polypeptide or a kinase compound using a surface stabilizer, such as a surfactant, in order to efficiently deliver the polypeptide or kinase compound inside a body through granulation into nanoparticles because the polypeptide or kinase compound is often poorly water soluble (see, for example, PTL 1 and PTL 2).

Moreover, it has been known that a medical component is efficient and effective on disease when the medical component has a particular particle diameter, in addition to that medical component is simply formed into nanoparticles. It has been known that, as seen with an enhanced permeation and retention effect (EPR effect), for example, neovascularity of an inflamed site of cancer tissues is incomplete, and therefore there are gaps of about several hundred nanometers between vascular endothelial cells around the inflamed site and nanoparticles a size of which is controlled to about 100 nm are accumulated on the cancer cells. Specifically, nanoparticles particle diameters of which are controlled to certain diameters are desired for a drug delivery system.

As a production method of nanoparticles, moreover, proposed is, for example, a method for using a liquid column resonance method in order to obtain particles having a certain particle size distribution (see, for example, PTL 3).

### CITATION LIST

### Patent Literature

PTL 1: Japanese Patent No. 4611641
PTL 2: Japanese Patent No. 4072057
PTL 3: Japanese Unexamined Patent Application Publication No. 2018-052922

### SUMMARY OF INVENTION

### Technical Problem

The present disclosure has an object to provide a nanoparticle having a desirable particle diameter suitable for filtration sterilization and applicable as a drug delivery system nanoparticle.

### Solution to Problem

According to one aspect of the present disclosure, a nanoparticle includes a poorly-water-soluble physiologically active compound, and an additive substance. A relative span factor (R.S.F) of the nanoparticle satisfies formula: 0<(R.S.F)≤1.0. A volume average particle diameter of the nanoparticle is 200 nm or less. The poorly-water-soluble physiologically active compound is covered with the additive substance.

### Advantageous Effects of Invention

The present disclosure can provide a nanoparticle having a desirable particle diameter suitable for filtration sterilization and applicable as a drug delivery system nanoparticle.

### BRIEF DESCRIPTION OF DRAWINGS

FIG. 1 is a cross-sectional view illustrating one example of a liquid column resonance droplet-ejecting unit.

FIG. 2 is a schematic view illustrating one example of an apparatus for producing a nanoparticle.

FIG. 3 is a schematic view illustrating another example of the apparatus for producing a nanoparticle.

FIG. 4A is a schematic view illustrating another example of the apparatus for producing a nanoparticle.

FIG. 4B is an enlarged view illustrating an area adjacent to a solution ejecting unit of the apparatus of FIG. 4A.

FIG. 5 is a schematic view illustrating another example of the apparatus for producing a nanoparticle.

### DESCRIPTION OF EMBODIMENTS

(Nanoparticle)

The nanoparticle of the present disclosure each includes a poorly-water-soluble physiologically active compound, and an additive substance. A relative span factor (R.S.F) of the nanoparticle satisfies formula: 0<(R.S.F)≤1.0. A volume average particle diameter of the nanoparticle is 200 nm or less. The poorly-water-soluble physiologically active compound is covered with the additive substance. The nanoparticle may further include other components according to the necessity.

The present inventors have conducted researches on a nanoparticle having a desirable particle diameter suitable for filtration sterilization, and applicable as a drug delivery system nanoparticle. As a result, the present inventors have found the following insights.

In the related art, nanoparticles formed of a material, such as polylactic acid glycolic acid, often have stable (homogeneous) particle diameters after granulation thereof. However, in case of the polypeptide or kinase inhibitor that is poorly water-soluble, there is a problem that it is difficult to form stable (homogeneous) nanoparticles.

As a result of the researches conducted by the present inventors, the present inventors have found that the poorly-water-soluble physiologically active compound can be formed into particles having a certain particle diameter and particle size distribution using a certain additive substance.

<Properties of Nanoparticle>

<<Volume Average Particle Diameter>>

The volume average particle diameter of the nanoparticles is 200 nm or less, preferably 5 nm or greater but 150 nm or less, more preferably 10 nm or greater but 110 nm or less, and even more preferably 10 nm or greater but 100 nm or less. When the volume average particle diameter of the nanoparticles is 200 nm or less, filtration sterilization can be performed simply without clogging a filter for filtration sterilization.

The filtration sterilization is a method for removing bacteria, such as microbes, present on a sterilization target through filtration, and typically a membrane filter having pores of 0.22 micrometers is used. Therefore, the particle in a sterilization target should be at least a nanoparticle. Specifically, the nanoparticle is, for example, a particle having a diameter of 5 nm or greater but less than 1,000 nm. In order to improve sterilization efficiency, it is necessary to produce a nanoparticle of 200 nm or less, more preferably 150 nm or less.

The volume average particle diameter of the nanoparticles can be measured, for example, by means of a high-concentration system particle size analyzer (“FPAR-1000,” obtained from Otsuka Electronics Co., Ltd.) according a dynamic light scattering method.

<<Relative Span Factor (R.S.F)>>

The relative span factor (R.S.F) of the nanoparticles satisfies the following formula (1).

0<(R.S.F)≤1.0 Formula (1)

(R.S.F) is defined by (D90−D10)/D50.
D90 is 90% in the cumulative volume from the side of small particles in the cumulative particle size distribution, D50 is 50% in the cumulative volume from the side of small particles in the cumulative particle size distribution, and D10 is 10% in the cumulative volume from the side of small particles in the cumulative particle size distribution.

As mentioned above, the (R.S.F) is 0<(R.S.F)≤1.0, and preferably 0<(R.S.F) 0.6. When the (R.S.F) is greater than 1.0, the number of particles that cannot pass through a sterilization filter increases, to thereby lower a sterilization rate.

The (R.S.F) can be measured, for example, by means of a high-concentration system particle size analyzer (“FPAR-1000,” obtained from Otsuka Electronics Co., Ltd.) according a dynamic light scattering method.

—Poorly-Water-Soluble Physiologically Active Compound—

The poorly-water-soluble compound is a compound a water/octanol partition coefficient (log P value) of which is 3 or greater.

The water/octanol partition coefficient (log P value) is a ratio between a concentration of a certain compound dissolved in a water phase and a concentration of the compound dissolved in an octanol phase in a two-phase system of water and octanol, and is typically represented by Log₁₀ (concentration of octanol phase/concentration of water phase).

As a measuring method of the water/octanol partition coefficient (log P value), any method known in the art can be used. Examples thereof include a method disclosed in JIS Z 7260-107.

The poorly-water-soluble physiologically active compound is not particularly limited and may be appropriately selected depending on the intended purpose, as long as the water/octanol partition coefficient (log P value) of the poorly-water-soluble physiologically active compound is 3 or greater. Examples thereof include a pharmaceutical compound. Examples of the pharmaceutical compound include a kinase inhibitor, and polypeptide.

Examples of the kinase inhibitor include gefitinib, erlotinib, osimertinib, bosutinib, vandetanib, alectinib, lorlatinib, abemaciclib, tyrphostin AG494, sorafenib, dasatinib, lapatinib, imatinib, motesanib, lestaurtinib, tandutinib dorsomorphin, axitinib, and 4-benzyl-2-methyl-1,2,4-thiadiazolidine-3,5-dione.

Examples of the polypeptide include cyclosporin, vancomycin, teicoplanin, and daptomycin.

Other examples of the poorly-water-soluble physiologically active compound include quercetin, testosterone, indomethacin, tranilast, and tacrolimus. Among the above-listed examples, the poorly-water-soluble physiologically active compound is preferably a kinase inhibitor or polypeptide. The poorly-water-soluble physiologically active compound comprised in the nanoparticle of the present invention includes any forms or derivatives suitable for the intended purpose of the nanoparticle. Examples of the form or derivative include, but are not limited to, pharmaceutically acceptable form or derivatives such as salts, solvates, stereoisomers, derivatives having protecting groups, and the like.

An amount of the poorly-water-soluble physiologically active compound is preferably 0.001% by mass or greater but 75% by mass or less, and more preferably 0.1% by mass or greater but 50% by mass or less relative to a total amount of the nanoparticles.

—Additive Substance—

The additive substance is not particularly limited and may be appropriately selected depending on the intended purpose, as long as the additive substance can suppress aggregation of the nanoparticles, or crystal growth thereof. Examples of the additive substance include polyethylene glycol fatty acid ester, sorbitan fatty acid ester, polyoxyethylene hydrogenated castor oil, polyoxyethylene alkyl ether, quaternary ammonium salt, lecithin, polyvinyl pyrrolidone, polyvinyl alcohol, glyceride, fatty acid, and steroid. Among the above-listed examples, polyethylene glycol fatty acid ester, sorbitan fatty acid ester, polyvinyl pyrrolidone, polyvinyl alcohol, glyceride, fatty acid, steroid, and phospholipid. Moreover, polyethylene glycol fatty acid ester, sorbitan fatty acid ester, and fatty acid are more preferable. Specifically, polyoxyl 40 stearate, polysorbate 80, and stearic acid are preferable. The above-listed examples may be used alone or in combination.

Since the poor solvent contains the additive substance, the additive substance covers a surface of the poorly-water-soluble physiologically active compound to make the poorly-water-soluble physiologically active compound being water soluble and therefore easily taken in a biological body. The covering is not limited as long as the poorly-water-soluble physiologically active compound becomes water soluble and can be easily taken in the biological body. The covering may be full coverage or partial coverage. Further, the additive substance can prevent the aggregation of nanoparticles, and can inhibit crystal growth of the poorly-water-soluble compound.

The location of the additive substance is not particularly limited. The additive substance may be located, for example, on the surface of particles of the poorly-water-soluble physiologically active substance.

Moreover, the additive substance is preferably present to cover surfaces of particles of the poorly-water-soluble physiologically active substance.

An amount of the additive substance is not particularly limited and may be appropriately selected depending on the intended purpose. For example, the amount of the additive substrate is preferably 50% by mass or less, more preferably 10% by mass or less, and even more preferably 5% by mass or less, relative to a total amount of the nanoparticles.

<Other Components>

The above-mentioned other components are not particularly limited and may be appropriately selected depending on the intended purpose.

(Pharmaceutical Composition)

The pharmaceutical composition comprises the nanoparticle and may further comprise other components, such as a dispersant, according to the necessity. The nanoparticle of the present application may function as a functional particle in the pharmaceutical composition.

The functional particle is not particularly limited and may be appropriately selected depending on the intended purpose. Examples of the functional particle include an immediate-release particle, a sustained-release particle, a pH-dependent-release particle, a pH-independent-release particle, an enteric-coated particle, a controlled-release-coated particle, and a nanocrystal-containing particle. The above-listed examples may be used alone or in combination.

A dosage form of the pharmaceutical composition is not particularly limited and may be appropriately selected depending on the intended purpose. Examples thereof include oral preparations, such as tablets (e.g., sugar-coated tablets, film-coated tablets, sublingual tablets, buccal tablets, and orally disintegrating tablets), pills, granules, powder, capsules (e.g., soft capsules, and microcapsules), syrup, emulsions, suspensions, and films (e.g., orally disintegrating films, and mucoadhesive buccal films). Other examples of the dosage forms according to different administration methods include parenteral preparations, such as injections, instillation, transdermal delivery agents (e.g., iontophoresis transdermal delivery agents), suppository, ointment, intranasal administration agents, intrapulmonary administration agents, and eye drops. Moreover, the pharmaceutical composition may be a controlled release preparation, such as a rapid-release preparation, or a sustained-release preparation (e.g., sustained-release microcapsules).

<Dispersant>

As the dispersant, a dispersant identical to the dispersant of the additive substance in the nanoparticle can be used. The dispersant is suitably used for dispersing the poorly-water-soluble physiologically active compound.

The dispersant may be a low-molecular-weight dispersant or a high-molecular-weight dispersant.

The low-molecular-weight dispersant is a compound having the weight average molecular weight of less than 15,000. The high-molecular-weight dispersant is a compound including covalent bonds between repeating units composed of one or more monomers and having the weight average molecular weight of 15,000 or greater.

The low-molecular-weight dispersant is not particularly limited as long as the dispersant is acceptable as a component utilized with physiologically active substance such as a pharmaceutical composition etc. and may be appropriately selected depending on the intended purpose. Examples thereof include lipids, saccharides, cyclodextrins, amino acids, and organic acid. The above-listed examples may be used alone or in combination.

The lipids are not particularly limited and may be appropriately selected depending on the intended purpose. Examples of the lipids include medium or long chain monoglyceride, diglyceride, or tri glyceride, phospholipid, vegetable oil (e.g., soybean oil, avocado oil, squalene oil, sesame oil, olive oil, corn oil, rapeseed oil, safflower oil, and sunflower oil), fish oil, seasoning oil, water-insoluble vitamins, fatty acids, mixtures thereof, and derivatives thereof. The above-listed examples may be used alone or in combination.

The saccharides are not particularly limited and may be appropriately selected depending on the intended purpose. Examples of the saccharides include glucose, mannose, idose, galactose, fucose, ribose, xylose, lactose, sucrose, maltose, trehalose, turanose, raffinose, maltotriose, acarbose, water-soluble cellulose, synthetic cellulose, sugar alcohols (e.g., glycerin, sorbitol, lactitol, maltitol, mannitol, xylitol, erythritol, and polyol), and derivatives thereof. The above-listed examples may be used alone or in combination.

<Other Components>

The above-mentioned other components are not particularly limited and may be appropriately selected depending on the intended purpose. The above-mentioned other components are preferably components usable in pharmaceutical compositions in the art.

Examples of the above-mentioned other components include an excipient, a flavoring agent, a disintegrating agent, a liquidizer, an adsorbent, a lubricant, an odor-masking agent, a perfume, a colorant, an anti-oxidant, a masking agent, an anti-static agent, and a humectant. The above-listed examples may be used alone or in combination.

(Method for Producing Nanoparticle)

The method for producing nanoparticle of the present disclosure includes ejecting a solution including a poorly-water-soluble physiologically active compound from an ejection outlet including one or more pores each having an inner diameter of 1.0 mm or less into a poor solvent for the poorly-water-soluble physiologically active compound including an additive substance. The method may further include other steps according to the necessity.

The method for producing a nanoparticle of the present disclosure is suitable as a production method of the nanoparticle of the present disclosure.

As a result of the researches conducted by the present inventors, the present inventors have found the following insights. That is, “particle diameters” and a “particle size distribution” of particles to be obtained can be highly accurately controlled by ejecting a solution containing a poorly-water-soluble physiologically active compound into a poor solvent for the poorly-water-soluble physiologically active compound containing an additive substance.

The method for producing a nanoparticle of the present disclosure preferably uses a crystallization method.

The crystallization method is a method where a solution is mixed with a poor solvent, where the solution is obtained by obtained by dissolving a poorly-water-soluble physiologically active compound that is a target for granulation in a good solvent. As a result, the poorly-water-soluble physiologically active compound is turned into the saturated state to precipitate the poorly-water-soluble physiologically active compound, which cannot be dissolved, to thereby granulate the poorly-water-soluble physiologically active compound.

—Solution—

The solution is not particularly limited and may be appropriately selected depending on the intended purpose, as long as the solution is a solution including at least the poorly-water-soluble physiologically active compound. Examples of the solution include a solution obtained by dissolving the poorly-water-soluble physiologically active compound in a good solvent for the poorly-water-soluble physiologically active compound.

——Poorly-Water-Soluble Physiologically Active Compound——

As the poorly-water-soluble physiologically active compound, the same poorly-water-soluble physiologically active compound for the nanoparticle of the present disclosure can be used. Therefore, descriptions thereof are omitted.

——Good Solvent——

The good solvent is not particularly limited and may be appropriately selected depending on the intended purpose, as long as the good solvent is a good solvent for the poorly-water-soluble physiologically active compound. Examples thereof include ethanol, methanol, acetone, acetnitrile, dioxane, dimethylsulfoxide, dimethylformamide, dichloromethane, dichloroethane, chloroform, chlorobenzene, toluene, methyl acetate, and ethyl acetate. Ethanol is particularly preferable. The above-listed examples may be used alone or in combination.

In the present disclosure, the “good solvent” is a solvent having high solubility of the poorly-water-soluble physiologically active compound. The “poor solvent” is a solvent having low solubility of the poorly-water-soluble physiologically active compound or a solvent that does not dissolve the poorly-water-soluble physiologically active compound.

For example, the “good solvent” and “poor solvent” can be determined with a mass of the poorly-water-soluble physiologically active compound dissolved in 100 g of a solvent at a temperature of 25 degrees Celsius. In the present disclosure, the “good solvent” is preferably a solvent that can dissolve 0.1 g or greater of the poorly-water-soluble physiologically active compound. On the other hand, the “poor solvent” is preferably a solvent that dissolves only 0.05 g or less of the poorly-water-soluble physiologically active compound.

A method for dissolving the poorly-water-soluble physiologically active compound in a good solvent for the poorly-water-soluble physiologically active compound is not particularly limited and may be appropriately selected depending on the intended purpose. For example, the poorly-water-soluble physiologically active compound may be added to a good solvent for the poorly-water-soluble physiologically active compound, or a good solvent for the poorly-water-soluble physiologically active compound may be added to the poorly-water-soluble physiologically active compound.

When the poorly-water-soluble physiologically active compound is dissolved in a good solvent for the poorly-water-soluble physiologically active compound, an auxiliary unit may be used. The auxiliary unit is not particularly limited. Examples thereof include a stirring unit, a shaking unit, and an ultrasonic wave treatment unit.

An amount of the poorly-water-soluble physiologically active compound in the solution is not particularly limited and may be appropriately selected depending on the intended purpose. The amount thereof as a concentration (amount) in a mixed solvent of acetone and ethanol is, for example, preferably 5.0% by mass or less, and more preferably 0.1% by mass or greater but 5.0% by mass or less. When the concentration thereof is 5.0% by mass or less, the resultant nanoparticles can be prevented from having an undesirable particle size distribution due to aggregations.

A particle diameter of a nanoparticle to be produced can be controlled at some degrees by adjusting the amount of the poorly-water-soluble physiologically active compound in the solution.

——Poor Solvent——

The poor solvent is not particularly limited and may be appropriately selected depending on the intended purpose. The poor solvent is preferably water. An additive substance is dispersed in the poor solvent of the present disclosure. When the poorly-water-soluble physiologically active compound is dripped in the poor solvent, therefore, particles of the poorly-water-soluble physiologically active compound are covered with the additive substance in the form of shells.

——Additive Substance——

The additive substance is identical to the additive substance in the nanoparticle of the present disclosure. Therefore, descriptions thereof are omitted.

The timing for adding the additive substance to the good solvent and the poor solvent is not particularly limited and may be appropriately selected depending on the intended purpose. In the case where particles are produced using the crystallization method, the additive substance may be dissolved in the good solvent as well as the poor solvent.

<<Ejection Hole>>

The ejection outlet is not particularly limited and may be appropriately selected depending on the intended purpose, as long as the ejection outlet includes a pore having an internal diameter of 1,000 micrometers or less.

The internal diameter is preferably 1.0 micrometer or greater but 1,000 micrometer or less, more preferably 1.0 micrometer or greater but 500 micrometers or less, and even more preferably 1.0 micrometer or greater but 50 micrometers or less.

When the pore is not a perfect circle, the pore may have an area equivalent to an area of a true circle having a diameter of 1,000 micrometers or less. Note that, the internal diameter of the ejection outlet is a value calculated as an area circle equivalent diameter.

The ejection outlet may be or may not be placed into the poor solvent. The ejection outlet is preferably placed into the poor solvent because the solution present at the ejection outlet is prevented from being dried and ejection failures caused due to the dried solution at the ejection outlet can be prevented. In other words, the ejection outlet is preferably in contact with the poor solvent.

The distance for inserting the ejection outlet in the poor solvent is not particularly limited and may be appropriately selected depending on the intended purpose. The distance is preferably 1.0 mm or greater but 10 mm or less, and more preferably 2.0 mm or greater but 5.0 mm or less. In other words, the ejection outlet is preferably immersed into the poor solvent by 1.0 mm or greater but 10 mm or less, and more preferably 2.0 mm or greater but 5.0 mm or less.

<<<Solution Ejecting Unit>>>

The ejection outlet is formed, for example, in the solution ejecting unit.

Examples of the solution ejecting unit includes the following units.

(i) A flat plate nozzle ejecting unit where pressure is applied to the solution to eject the solution from pores made in a flat plate, such as an inkjet nozzle.

(ii) An ejecting unit where pressure is applied to the solution to eject the solution from pores of irregular shapes, such as a SPG film.

(iii) An ejecting unit where vibrations are applied to the solution to eject the solution from pores as liquid droplets.

Examples of the (iii) ejecting unit include a membrane vibration ejecting unit, a Rayleigh breakup ejecting unit, a liquid vibration ejecting unit, and a liquid column resonance ejecting unit. Moreover, ejection may be performed by applying pressure to the solution at the same time, and the above-listed units may be used in combination.

Examples of the membrane vibration ejecting unit include an ejecting unit disclosed in Japanese Unexamined Patent Application Publication No. 2008-292976.

Examples of the Rayleigh breakup ejecting unit include an ejecting unit disclosed in Japanese Patent No. 4647506.

Examples of the liquid vibration ejecting unit include an ejecting unit disclosed in Japanese Unexamined Patent Application Publication No. 2010-102195.

Among the above-listed examples, preferable is a unit where pressure is applied to a liquid column resonance ejecting unit using a liquid column resonance method.

The liquid column resonance method is not particularly limited and may be appropriately selected depending on the intended purpose. Examples thereof include: a method where vibrations are applied to a solution stored in a liquid-column-resonance liquid chamber to form standing waves due to liquid column resonance to eject the solution from the ejection outlet formed in the amplification direction of the standing waves in the regions that correspond to anti-nodes of the standing waves.

The liquid column resonance method can be suitably performed by the below-described liquid column resonance droplet-ejecting unit.

<<Liquid-Flowing Treatment>>

The liquid-flowing treatment is not particularly limited and may be appropriately selected depending on the intended purpose, as long as the liquid-flowing treatment is a treatment for making the liquid flow when the solution is ejected into the liquid that is the poor solvent. The flow speed of the liquid is preferably 0.3 m/s or greater, and more preferably 1.0 m/s or greater.

Cohesion of the nanoparticles can be prevented by performing the liquid-flowing treatment.

Examples of a liquid-flowing unit configured to make the liquid flow include a stirring member configured to stir the liquid. The stirring member is not particularly limited and may be appropriately selected depending on the intended purpose. Examples of the stirring member include a stirring blade.

<<Liquid-Circulating Treatment>>

During the step for ejecting the solution, the solution is preferably ejected from the ejection outlet into the liquid that is circulated in view of prevention of cohesion between nanoparticles.

To this end, a liquid-circulating treatment to circulate the liquid is preferably performed.
For example, a pump is used as a circulating member to circulate the liquid inside the poor solvent storage member having a circulation path during the liquid-circulating treatment.

<<<Good Solvent Removal Treatment>>>

In the case where the liquid is circulated, the good solvent for the poorly-water-soluble physiologically active compound is accumulated in the liquid. When the good solvent is accumulated in the liquid, cohesion between nanoparticles tend to occur. Therefore, a good solvent removal treatment where the good solvent included in the circulated liquid is removed is preferably performed.

The good solvent removal treatment is not particularly limited and may be appropriately selected depending on the intended purpose, as long as the good solvent can be removed from the liquid. Examples thereof include a method where the good solvent is evaporated by heating the liquid or decompressing the liquid to remove the good solvent from the liquid.

<Other Steps>

Examples of other steps include a good solvent removing step, a filtration sterilization step, and a poor solvent removing step.

<<Good Solvent Removing Step>>

The good solvent removing step is not particularly limited and may be appropriately selected depending on the intended purpose, as long as the good solvent removing step is a step including removing the good solvent from the produced nanoparticles. Examples thereof include a method where a decompression treatment is performed on the liquid including the nanoparticles to evaporate only the good solvent for the poorly-water-soluble physiologically active compound to obtain a suspension liquid including the nanoparticles.

<<Filtration Sterilization Step>>

The filtration sterilization step is not particularly limited and may be appropriately selected depending on the intended purpose, as long as the filtration sterilization step is a step including performing filtration of the nanoparticle suspension liquid after the good solvent removing step using a sterilization filter.

The nanoparticle suspension liquid provided to the filtration may be diluted or may not be diluted with the poor solvent.

Ultrasonic waves are preferably applied to the nanoparticle suspension liquid before performing the filtration. As a result, aggregations of the nanoparticles in the suspension liquid are disassembled and the nanoparticles are easily passed through the filter.

The sterilization filter is not particularly limited and may be appropriately selected depending on the intended purpose. Examples thereof include a nylon membrane filter.

The filtration rating of the sterilization filter is not particularly limited and may be appropriately selected depending on the intended purpose. The filtration rating thereof is preferably 0.1 micrometers or greater but 0.45 micrometers or less.

A commercial product of the sterilization filter may be used. Examples of the commercial product include LifeASSUR™ nylon membrane filter cartridge (filtration rating: 0.1 micrometers).

A method for removing the poor solvent is not particularly limited and may be appropriately selected depending on the intended purpose. Examples thereof include a method where the poor solvent is removed by the filtration step. The particles, from which the poor solvent has been removed, are dried to thereby obtain the nanoparticle of the present disclosure.

(Apparatus for Producing Nanoparticle)

The apparatus for producing a nanoparticle of the present disclosure includes a solution storage container configured to store a solution, in which the poorly-water-soluble physiologically active compound is dissolved, and a solution ejecting unit which is connected to the solution storage container and includes one or more ejection outlets each having a pore having an inner diameter of less than 1,000 micrometers. The apparatus may further include a poor solvent storage member configured to store a liquid that is a poor solvent for the poorly-water-soluble physiologically active compound, a liquid-flowing unit, and other members according to the necessity.

The apparatus for producing a nanoparticle will be described hereinafter. The terms identical to the terms described in the method for producing a nanoparticle of the present disclosure have the same meaning unless there are descriptions of the terms below. Examples and preferable embodiments of such terms are the same as the examples and preferable embodiments of the terms described in the method for producing a nanoparticle.

<Solution Storage Container>

The solution storage container is not particularly limited and may be appropriately selected depending on the intended purpose, as long as the solution storage container is a container configured to store therein a solution. The solution storage container may have flexibility or may not have flexibility.

A material of the solution storage container is not particularly limited and may be appropriately selected depending on the intended purpose. For example, the solution storage container may be formed of a resin, or may be formed of a metal.

A structure of the solution storage container is not particularly limited and may be appropriately selected depending on the intended purpose. For example, the solution storage container may be a sealed container or unsealed container.

In the solution, the poorly-water-soluble physiologically active compound is dissolved in a good solvent for the poorly-water-soluble physiologically active compound.

<Solution Ejecting Unit>

The solution ejecting unit is not particularly limited and may be appropriately selected depending on the intended purpose, as long as the solution ejection unit has one or more ejection outlets each having a pore having an inner diameter of less than 1,000 micrometers.

The solution ejecting unit is connected to the solution storage container. A method for connecting between the solution ejecting unit and the solution storage container is not particularly limited and may be appropriately selected depending on the intended purpose, as long as the solution can be supplied from the solution storage container to the solution ejecting unit. Examples thereof include pipes, and tubes.

The solution ejecting unit preferably includes a vibration applying member configured to apply vibrations to the solution.

The vibrations are not particularly limited and may be appropriately selected depending on the intended purpose. For example, the frequency is preferably 1 kHz or greater, more preferably 150 kHz or greater, and even more preferably 300 kHz or greater but 500 kHz or less. When the vibrations are 1 kHz or greater, liquid columns ejected from the ejection outlets can be formed into liquid droplets with good reproducibility. When the vibrations are 150 kHz or greater, production efficiency can be improved.

Examples of the solution ejecting unit including the vibration applying member include an inkjet. Examples of the inkjet include units using a liquid column resonance method, a membrane vibration method, a liquid vibration method, a Rayleigh breakup method, a thermal method, etc.

<Poor Solvent Storage Member>

The poor solvent storage member is not particularly limited and may be appropriately selected depending on the intended purpose without any limitation, as long as the poor solvent storage member is a member configured to store a poor solvent for the poorly-water-soluble physiologically active compound. The poor solvent storage member may have flexibility or may not have flexibility.

A material of the poor solvent storage member is not particularly limited and may be appropriately selected depending on the intended purpose. For example, the poor solvent storage member may be formed of a resin, or may be formed of a metal.

The poor solvent in the poor solvent storage member may be stirred or may not be stirred when nanoparticles are produced, but the poor solvent is preferably stirred.

The ejection outlet of the solution ejecting unit may be or may not be placed in the poor solvent in the poor solvent storage member. The ejection outlet is preferably placed into the poor solvent because the solution present at the ejection outlet is prevented from being dried and ejection failures caused due to the dried solution at the ejection outlet can be prevented. In other words, the ejection outlet of the solution ejecting unit is preferably in contact with the poor solvent in the poor solvent storage member.

The distance for inserting the ejection outlet in the poor solvent in the poor solvent storage member is not particularly limited and may be appropriately selected depending on the intended purpose. The distance is preferably 1.0 mm or greater but 10 mm or less, and more preferably 2.0 mm or greater but 5.0 mm or less. In other words, the ejection outlet of the solution ejecting unit is preferably immersed into the poor solvent in the poor solvent storage member by 1.0 mm or greater but 10 mm or less, and more preferably 2.0 mm or greater but 5.0 mm or less.

The poor solvent storage member preferably has a circulation path capable of circulating the liquid. The circulation path capable of circulating the liquid may be, for example, a circulation path composed only of piping, or a circulation path including piping and tanks.

<<Good Solvent Removing Member>>

In the case where the liquid is circulated, the good solvent for the poorly-water-soluble physiologically active compound is accumulated in the liquid. When the good solvent is accumulated in the liquid, cohesion between nanoparticles tend to occur. Therefore, a good solvent removing member configured to remove the good solvent included in the circulated liquid is preferably arranged.

The good solvent removing member is not particularly limited and may be appropriately selected depending on the intended purpose, as long as the good solvent removing member is capable of removing the good solvent from the liquid. Examples thereof include a heating unit configured to heat the liquid, and a decompressing unit configured to decompress the liquid. Use of the heating unit, or the decompressing unit, or both can evaporate the good solvent to remove the good solvent from the liquid.

<Fluid-Flowing Unit>

The liquid-flowing unit is not particularly limited and may be appropriately selected depending on the intended purpose, as long as the liquid-flowing unit is a unit capable of making the liquid flow, where the liquid is the poor solvent in the poor solvent storage member. Examples thereof include a stirring member configured to stir the liquid.

Use of the liquid-flowing unit can prevent cohesion of the nanoparticles.

The nanoparticle of the present disclosure and particles obtained by the method for producing a nanoparticle of the present disclosure and the apparatus for producing a nanoparticle are particles suitable for filtration sterilization. The filtration sterilization is a method where bacteria, such as microbes, present on a sterilization target is removed by filtration, and typically uses a membrane filter having an opening size of 0.22 micrometers. Therefore, it is difficult to sufficiently pass nanoparticles of a pharmaceutical composition compound having particle diameters of 200 nm or greater through a filter for filtration sterilization.

The liquid column resonance droplet-ejecting unit, which is one embodiment of the solution ejecting unit, will be described below.

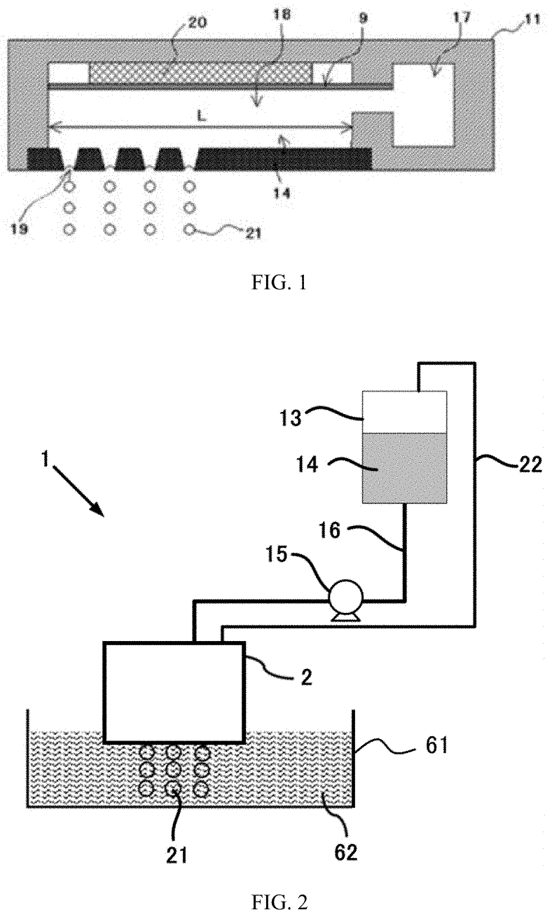

FIG. 1 is a schematic cross-sectional view of the liquid column resonance droplet-ejecting unit 11. The liquid column resonance droplet-ejecting unit 11 includes a common liquid supplying path 17 and a liquid-column-resonance liquid chamber 18. The liquid-column-resonance liquid chamber 18 is connected to the common liquid supplying path 17 disposed on one of wall surfaces at both ends in a longitudinal direction. Moreover, the liquid-column-resonance liquid chamber 18 includes an ejection outlet 19 and a vibration generating unit 20. The ejection outlet 19 is configured to eject liquid droplets 21 and arranged on one of the wall surfaces connected to the wall surfaces at the both ends. The vibration generating unit 20 is configured to generate high frequency vibrations to form liquid column resonance standing waves. Note that, a high frequency power source, which is not illustrated, is coupled to the vibration generating unit 20. Moreover, a flow channel 12 may be disposed. The flow channel 12 is configured to supply an air flow for transporting liquid droplets 21 ejected from the liquid column resonance ejecting unit 11.

The solution 14 is passed through a liquid supply pipe and introduced into the common liquid supplying path 17 of the liquid column resonance liquid droplet forming unit by a liquid-circulating pump that is not illustrated, and then is supplied to the liquid-column-resonance liquid chamber 18 of the liquid column resonance droplet-ejecting unit 11. Within the liquid-column-resonance liquid chamber 18 charged with the solution 14, a pressure distribution is formed by liquid column resonance standing waves generated by the vibration generating unit 20. Then, liquid droplets 21 are ejected from the ejection outlet 19 disposed in the regions that correspond to anti-nodes of the standing waves where the regions are the sections where the amplitude of the liquid column resonance standing waves is large and pressure displacement is large. The regions corresponding to anti-nodes of the standing waves owing to the liquid column resonance are regions other than nodes of the standing waves. The regions are preferably regions each having sufficiently large amplitude enough to eject the liquid through the pressure displacement of the standing waves, are more preferably regions having a width corresponding to ±¼ of a wavelength from a position of a local maximum amplitude of a pressure standing wave (i.e., a node of a velocity standing wave) toward positions of a local minimum amplitude.

Even when there are a plurality of openings of the ejection outlet, substantially uniform liquid droplets can be formed from the openings as long as the openings of the ejection outlet are disposed in the regions corresponding to the anti-nodes of the standing waves. Moreover, ejection of the liquid droplets can be performed efficiently, and clogging of the ejection outlet is unlikely to occur. Note that, the solution 14 passed through the common liquid supplying path 17 travels through a liquid returning pipe (not illustrated) to be returned to the solution 14. Once the amount of the solution 14 inside the liquid-column-resonance liquid chamber 18 is reduced by ejection of the liquid droplets 21, a flow rate of the solution 14 supplied from the column liquid supplying path 17 by suction power generated by the action of the liquid column resonance standing waves inside the liquid-column-resonance liquid chamber 18. As a result, the liquid-column-resonance liquid chamber 18 is refilled with the solution 14. When the liquid-column-resonance liquid chamber 18 is refilled with the solution 14, the flow rate of the solution 14 passed through the common liquid supplying path 17 returns to the previous flow rate.

The liquid-column-resonance liquid chamber 18 of the liquid column resonance droplet-ejecting unit 11 is formed by joining frames with each other. The frames are formed of materials having high stiffness to the extent that a resonance frequency of the liquid is not influenced at a driving frequency (e.g., metals, ceramics, and silicones). As illustrated in FIG. 1, a length L between the wall surfaces at the both ends of the liquid-column-resonance liquid chamber 18 in a longitudinal direction is determined based on the principle of the liquid column resonance described below. Moreover, a plurality of the liquid-column-resonance liquid chambers 18 are preferably disposed per one liquid droplet forming unit 10 in order to drastically improve productivity. The number of the liquid-column-resonance liquid chambers 18 is not particularly limited. The number thereof is preferably 1 or greater but 2,000 or less. The common liquid supplying-path 17 is coupled to and connected to a path for supplying the liquid for each liquid-column-resonance liquid chamber. The common liquid supplying path 17 is connected to a plurality of the liquid-column-resonance liquid chambers 18.

Moreover, the vibration generating unit 20 of the liquid column resonance droplet-ejecting unit 11 is not particularly limited as long as the vibration generating unit 20 is driven at a predetermined frequency. The vibration generating unit is preferably formed by attaching a piezoelectric material onto an elastic plate 9. The frequency is preferably 150 kHz or greater, more preferably 300 kHz or greater but 500 kHz or less from the viewpoint of productivity. The elastic plate constitutes a portion of the wall of the liquid-column-resonance liquid chamber in a manner that the piezoelectric material does not come into contact with the liquid. The piezoelectric material may be, for example, piezoelectric ceramics such as lead zirconate titanate (PZT), and is typically often laminated due to a small displacement amount. Other examples of the piezoelectric material include piezoelectric polymers (e.g., polyvinylidene fluoride (PVDF)) and monocrystals (e.g., crystal, LiNbO₃, LiTaO₃, and KNbO₃). The vibration generating unit 20 is preferably disposed per liquid-column-resonance liquid chamber in a manner that the vibration generating unit 20 can individually control each liquid-column-resonance liquid chamber. It is preferable that the liquid-column-resonance liquid chambers be individually controlled via the elastic plates by partially cutting a block-shaped vibration member, which is formed of one of the above-described materials, according to geometry of the liquid-column-resonance liquid chambers.

Moreover, a plurality of openings are formed in the ejection outlet 19. In view of productivity, preferably employed is a structure where the ejection outlet 19 is disposed along the width direction of the liquid-column-resonance liquid chamber 18. Moreover, the frequency of the liquid column resonance is desirably appropriately determined with checking ejection of liquid droplets, because the frequency of the liquid column resonance varies depending on the arrangement of opening of the ejection outlet 19.

The paragraphs [0011] to [0020] of Japanese Unexamined Patent Application Publication No. 2011-194675 can be referred for the mechanism for forming liquid droplets according to liquid column resonance.

Next, an example of the apparatus for producing a nanoparticle according to the present disclosure will be described with reference to a drawing.

FIG. 2 is a schematic view illustrating one example of the apparatus for producing a nanoparticle. The apparatus for producing a nanoparticle 1 mainly includes a solution storage container 13, a solution ejecting unit 2, and a poor solvent storage member 61. To the solution ejecting unit 2, the solution storage container 13 and the liquid-circulating pump 15 are connected. The solution storage container 13 is configured to store the solution 14. The liquid-circulating pump 15 is configured to supply the solution stored in the solution storage container 13 to the solution ejecting unit 2 via the liquid supply tube 16. Moreover, the liquid-circulating pump 15 is configured to pressure feed the solution inside the liquid supply tube 16 to return to the solution storage container 13 via a liquid returning tube 22. Therefore, the solution 14 can be supplied to the solution ejection unit 2 at any time.

The solution ejecting unit 2 includes, for example, the liquid column resonance droplet-ejecting unit 11 illustrated in FIG. 1.

The solution 14 is ejected as liquid droplets 21 from the solution ejecting unit 2 into the poor solvent 62 stored in the poor solvent storage member 61.

Since the liquid droplets 21 are in contact with the poor solvent 62, the solution is diffused and therefore the poorly-soluble physiologically active substance is brought into contact with the poor solvent. As a result, the solubility is reduced, and the poorly-soluble physiologically active substance is crystallized to yield nanoparticles.

Next, another example of the apparatus for producing a nanoparticle of the present disclosure will be described with reference to a drawing.

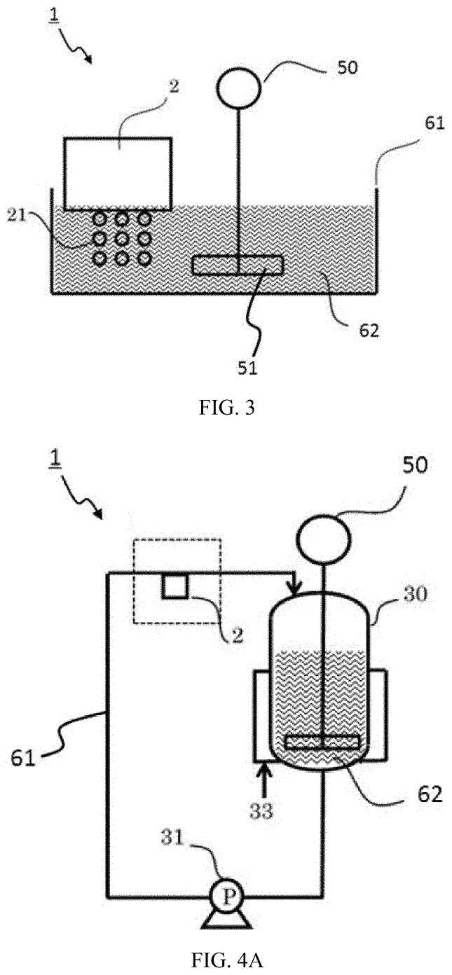

FIG. 3 is an example of the apparatus for producing a nanoparticle where the apparatus includes a stirring member.

The apparatus for producing a nanoparticle 1 of FIG. 3 is a schematic view illustrating a case where a solution is ejected into a poor solvent 62 in a poor solvent storage member 61 that is a glass container. An ejection part of the solution ejecting unit 2 is configured to eject the solution into the poor solvent 62 in the state where the ejection part is immersed in the poor solvent 62.

The apparatus for producing a nanoparticle 1 of FIG. 3 includes a stirring member 50 including a stirring blade 51. The stirring blade 51 is immersed in the poor solvent 62 in the poor solvent storage member 61.

When the solution is ejected into the poor solvent 62 by the solution ejecting unit 2, the stirring blade 51 is rotated to stir the poor solvent 61. As a result, cohesion between nanoparticles formed of the liquid droplets 21 can be prevented.

Next, another example of the apparatus for producing a nanoparticle of the present disclosure will be described with reference to drawings.

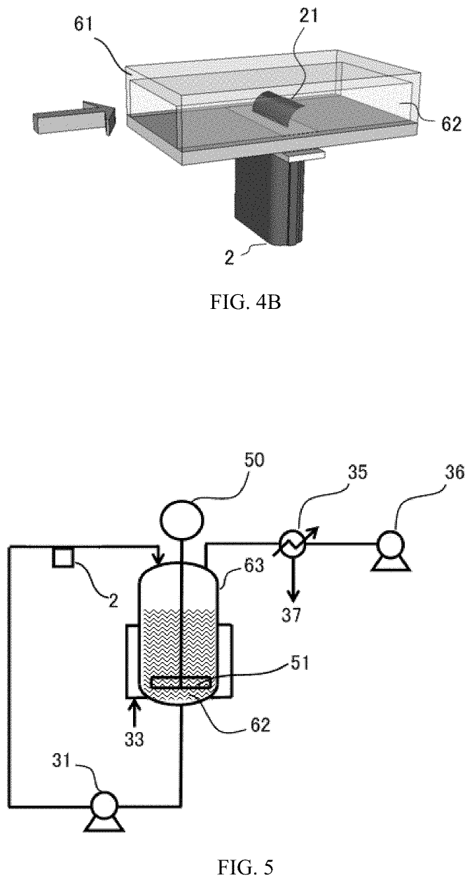

As a method for preventing cohesion between nanoparticles formed by bringing the solution into contact with the poor solvent, to apply a flow of the poor solvent to the ejection part of the solution ejecting unit is the most preferable. FIGS. 4A and 4B are preferable in this regard.

FIG. 4A is a schematic view illustrating one example of the apparatus for producing a nanoparticle where the apparatus can apply a flow of the poor solvent to the ejection part of the solution ejecting unit.

The apparatus for producing a nanoparticle of FIG. 4A includes a solution ejecting unit 2, a poor solvent storage member 61, a stirring member 50, and a pump 31.

The poor solvent storage member 61 includes a circulation path capable of circulating the liquid. As a part of the poor solvent storage member 62, a tank 63 is disposed in the middle of the circulation path.

FIG. 4B is an enlarged view of an area adjacent to the solution ejecting unit 2 (section marked with a broken-line) of FIG. 4A.

The poor solvent 62 in the tank 63 is circulated inside the poor solvent storage member 61 via the solution ejecting unit 2 by the pump 31. At this time, the solution is ejected from the ejection outlet of the solution ejecting unit 2 into the poor solvent 62.
Cohesion of nanoparticles formed of the liquid droplets 21 is prevented by imparting a flow to the liquid that is the poor solvent 62. The flow rate of the poor solvent 62 at the ejection outlet of the solution ejecting unit 2 is preferably 0.3 m/s or greater, and more preferably 1.0 m/s or greater.
The tank 63 includes a stirring member 50 including a stirring blade 51. Cohesion of the nanoparticles can be prevented by stirring the liquid that is the poor solvent 62 with the stirring blade 51.

Next, another example of the apparatus for producing a nanoparticle will be described with reference to a drawing.

When an amount of the good solvent in the liquid increases, occurrences of cohesion of nanoparticles increase, and particle diameters thereof tend to be large. In order to prevent generation of particles having large particle diameters, the good solvent is preferably removed from the liquid to maintain the amount of the good solvent in the liquid low.

FIG. 5 is a schematic view illustrating one example of the apparatus for producing a nanoparticle including a good solvent removing member configured to remove the good solvent.

The apparatus for producing a nanoparticle of FIG. 5 includes a solution ejecting unit 2, a poor solvent storage member 61, a stirring member 50, a pump 31, and a heating unit 33 and decompression unit 36 (vacuum pump) serving as a good solvent removing member.

The structure of the area adjacent to the solution ejecting unit 2 is identical to FIGS. 4A and 4B.

The poor solvent storage member 61 is a circulation path capable of circulating the liquid. As a part of the poor solvent storage member 61, a tank 63 is disposed in the middle of the circulation path.

The poor solvent 62 in the tank 63 is circulated in the poor solvent storage member 61 via the solution ejecting unit 2 by the pump 31. The solution is ejected from the ejection outlet of the solution ejecting unit 2 into the poor solvent 62. Cohesion of nanoparticles formed of the liquid droplets 21 is prevented by imparting a flow to the liquid that is the poor solvent 62.

Moreover, the good solvent included in the liquid that is the poor solvent 62 can be removed because the tank 63 includes the heating unit 33 and the decompression unit 36. For example, the liquid that is the poor solvent 62 is decompressed by the decompression unit 36 with heating the liquid using the heating unit 33. As a result, the good solvent having a boiling point lower than a boiling point of the poor solvent is evaporated. The evaporated good solvent is condensed by a condenser 35 and is collected through a collecting tube 37.

The nanoparticles produced by the method and apparatus for producing a nanoparticle of the present disclosure have the following properties.

<Properties of Nanoparticles>

<<Volume Average Particle Diameter>>

The volume average particle diameter of the nanoparticles is 100 nm or less, preferably 10 nm or greater but 50 nm or less, more preferably 10 nm or greater but 40 nm or less, and particularly preferably 10 nm or greater but 30 nm or less.

The volume average particle diameter of the nanoparticles can be measured by means of a high-concentration system particle size analyzer (“FPAR-1000,” obtained from Otsuka Electronics Co., Ltd.) according a dynamic light scattering method.

### EXAMPLES

The present disclosure will be described more detail by way of Examples. However, the present disclosure should not be construed as being limited to these Examples.

### Example 1

<Preparation of Solution>

In ethanol (obtained from FUJIFILM Wako Pure Chemical Corporation) serving as a good solvent, cyclosporin A (obtained from Tokyo Chemical Industry Co., Ltd.) serving as a poorly-water-soluble physiologically active compound and stearic acid (obtained from Tokyo Chemical Industry Co., Ltd.) were dissolved to give a concentration of 3% by mass of the cyclosporin A and a concentration of 0.06% by mass of the stearic acid, to thereby prepare a cyclosporin A solution.

<Granulation of Nanoparticles>

The prepared cyclosporin A solution (5 g) was ejected by means of an apparatus for producing a nanoparticle at the rotational speed of the stirring member being 200 rpm under the following ejection conditions, to thereby obtain a liquid in which particles of the cyclosporin A were granulated. The apparatus included a stirring member illustrated in FIG. 3 and a liquid column resonance unit illustrated in FIG. 1. Note that, the poor solvent storage member 24 formed of glass illustrated in FIG. 3 was charged with 100 g of ion-exchanged water.

—Ejection Conditions—

Nozzle diameter: 5.0 micrometers

Liquid feeding pressure: 0.05 MPa

Solution ejecting unit: liquid column resonance

Driving frequency: 390 kHz

Applying voltage to piezoelectric material: 5.0 V

<Removal of Good Solvent>

Next, the good solvent (ethanol) was removed by a decompression treatment for 24 hours at −50 kPa with stirring at 200 rpm, to thereby obtain a suspension liquid of the particles of the cyclosporin A.

<Evaluation of Particle Size Distribution>

The volume average particle diameter and (R.S.F) of the obtained suspension liquid of the particles of the cyclosporin A were measured by means of a high-concentration system particle size analyzer (“FPAR-1000,” obtained from Otsuka Electronics Co., Ltd.) according to a dynamic light scattering method. The results are presented in Table 1. The solid content of the particles in the suspension liquid of the particles of the cyclosporin A provided to the measurement was adjusted to 0.1% by mass. The volume average particle diameter (nm) was determined according to the CINTIN algorithm with a calmative time per measurement being 180 seconds. The average value of three measurement values was determined as the volume average particle diameter (nm) in the present disclosure. Note that, the measured volume average particle diameter and (R.S.F) were evaluated based on the following evaluation criteria.

(Evaluation Criteria: Volume Average Particle Diameter)

Excellent: The volume average particle diameter was 5 nm or greater but 150 nm or less.

Good: The volume average particle diameter was greater than 150 nm but 200 nm or less.

Poor: The volume average [article diameter was greater than 200 nm.

(Evaluation Criteria: (R.S.F))

Excellent: 0<(R.S.F)≤0.6

Good: 0.6<(R.S.F)≤1.0

Poor: 1.0<(R.S.F)

<Evaluation of Sterilization Rate>

Filtration sterilization was performed on the prepared nanoparticle suspension liquid of the cyclosporin A using a nylon membrane filter for sterilization having a pore size of 0.2 micrometers (product name: PSA, obtained from 3M). Moreover, the filtrate obtained after the filtration sterilization was sufficiently dried in a drying furnace of 50 degrees Celsius, and a weight of the remained particles of the cyclosporin A was measured to calculate a sterilization rate. The result is presented in Table 1. Note that, the sterilization rate was calculated according to the following formula, and the evaluation was performed based on the sterilization rate.

Sterilization rate (%)=[(weight of nanoparticles of cyclosporin A dried after filtration)/(weight of solids of cyclosporin A added to suspension liquid before filtration)]×100

(Evaluation Criteria: Sterilization Rate (%))

Excellent: The sterilization rate was 90% or greater.

Good: The sterilization rate was 70% or greater but less than 90%.

Poor: The sterilization rate was less than 70%.

### Example 2

Particles of alectinib were produced in the same manner as in Example 1, except that the poorly-water-soluble physiologically active compound was changed from the cyclosporin A to alectinib (obtained from Selleck Chemicals), the stearic acid was changed to dioleoylphosphatidylcholine (product name: DOPC, obtained from FUJIFILM Wako Pure Chemical Corporation), the good solvent was changed from the ethanol to dimethyl sulfoxide (product name: DMSO, obtained from FUJIFILM Wako Pure Chemical Corporation), and a liquid obtained by dissolving 0.2% by mass of polyvinyl pyrrolidone (PVP-K30, obtained from Tokyo Chemical Industry Co., Ltd.) in 99.8% by mass of ion-exchanged water was placed in the poor solvent storage member 24 formed of glass illustrated in FIG. 3. The volume average particle diameter and (R.S.F) were measured and the sterilization rate was evaluated in the same manner as in Example 1. The conditions are presented in Table 1 and the results are presented in Table 2.

### Example 3

Particles of tranilast were obtained in the same manner as in Example 1, except that the poorly-water-soluble physiologically active compound was changed from the cyclosporin A to tranilast (obtained from Tokyo Chemical Industry Co., Ltd.) and Additive substance 1 was changed from stearic acid to polyoxyl 40 stearate (obtained from Nikko Chemicals Co., Ltd.). The volume average particle diameter and (R.S.F) were measured and the sterilization rate was evaluated in the same manner as in Example 1. The conditions are presented in Table 1 and the results are presented in Table 2.

### Example 4

Particles of cyclosporin A were obtained in the same manner as in Example 1, except that the stearic acid was changed to lecithin (obtained from Tokyo Chemical Industry Co., Ltd.), the system of the solution ejecting unit was changed from the liquid column resonance to a system where the solution was ejected from a flat plate nozzle without applying vibration (ejection speed: 18 g/min, driving system: pushing by liquid feeding pressure). The volume average particle diameter and (R.S.F) were measured and the sterilization rate was evaluated in the same manner as in Example 1. The conditions are presented in Table 1 and the results are presented in Table 2.

### Example 5

Particles of alectinib were obtained in the same manner as in Example 2, except that the dioleoylphosphatidylcholine was changed to polysorbate 80 (obtained from Nikko Chemicals Co., Ltd.), the polyvinyl pyrrolidone was changed to polyvinyl alcohol (PVA, obtained from FUJIFILM Wako Pure Chemical Corporation), and the system of the solution ejection unit was changed from the liquid column resonance to a TEFLON tube having an inner diameter of 1.0 mm (ejection speed: 300 g/min, driving system: pushing by liquid feeding pressure). The volume average particle diameter and (R.S.F) were measured and the sterilization rate was evaluated in the same manner as in Example 1. The conditions are presented in Table 1 and the results are presented in Table 2.

### Comparative Example 1

Particles of cyclosporin A were obtained in the same manner as in Example 1, except that the stearic acid was not added. The volume average particle diameter and (R.S.F) were measured and the sterilization rate was evaluated in the same manner as in Example 1. The conditions are presented in Table 1 and the results are presented in Table 2.

### Comparative Example 2

Particles of alectinib were obtained in the same manner as in Example 5, except that the polysorbate 80 and the polyvinyl alcohol were not added. The volume average particle diameter and (R.S.F) were measured and the sterilization rate was evaluated in the same manner as in Example 1. The conditions are presented in Table 1 and the results are presented in Table 2.

TABLE 1

| | | Poorly-water-soluble | | | | | |
|---|---|---|---|---|---|---|---|
| | | physiologically | Good | Poor | Additive | Additive | Solution |
| | | active compound | solvent | solvent | substance 1 | substance 2 | ejecting unit |
| Example | 1 | cyclosporin A | ethanol | ion- | stearic acid | — | liquid column |
| | | | | exchanged | | | resonance |
| | | | | water | | | |
| | 2 | alectinib | DMSO | ion- | DOPC | PVP | liquid column |
| | | | | exchanged | | | resonance |
| | | | | water | | | |
| | 3 | tranilast | ethanol | ion- | polyoxyl 40 | — | liquid column |
| | | | | exchanged | stearate | | resonance |
| | | | | water | | | |
| | 4 | cyclosporin A | ethanol | ion- | lecithin | — | flat plate |
| | | | | exchanged | | | nozzle |
| | | | | water | | | |
| | 5 | alectinib | DMSO | ion- | polysorbate 80 | PVA | tube |
| | | | | exchanged | | | |
| | | | | water | | | |
| Comparative | 1 | cyclosporin A | ethanol | ion- | — | — | liquid column |
| Example | | | | exchanged | | | resonance |
| | | | | water | | | |
| | 2 | alectinib | DMSO | ion- | — | — | tube |
| | | | | exchanged | | | |
| | | | | water | | | |

TABLE 2

| | | Evaluation results | | | | | |
|---|---|---|---|---|---|---|---|
| | | Average volume- | | | | | |
| | | based particle | | | | Sterilization | |
| | | diameter (nm) | | R.S.F | | rate (%) | |
| | | | Evaluation | | Evaluation | | Evaluation |
| Example | 1 | 96 | Excellent | 0.45 | Excellent | 94.2 | Excellent |
| | 2 | 84 | Excellent | 0.48 | Excellent | 95.1 | Excellent |
| | 3 | 102 | Excellent | 0.52 | Excellent | 94.6 | Excellent |
| | 4 | 131 | Excellent | 0.66 | Good | 80.2 | Good |
| | 5 | 168 | Good | 0.86 | Good | 72.3 | Good |
| Compara- | 1 | 38,834 | Poor | 1.48 | Poor | 3.3 | Poor |
| tive | 2 | 63,508 | Poor | 1.04 | Poor | 1.7 | Poor |
| Example | | | | | | | |

For example, embodiments of the present disclosure are as follows.

<1> A nanoparticle including:

a poorly-water-soluble physiologically active compound; and

an additive substance,

wherein a relative span factor (R.S.F) of the nanoparticle satisfies formula: 0<(R.S.F)≤1.0,

a volume average particle diameter of the nanoparticle is 200 nm or less, and

the poorly-water-soluble physiologically active compound is covered with the additive substance.

<2> The nanoparticle according to <1>,

wherein the volume average particle diameter is 5 nm or greater but 150 nm or less.

<3> The nanoparticle according to <1> or <2>,

wherein the (R.S.F) satisfies: 0<(R.S.F)≤0.6.

<4> The nanoparticle according to any one of <1> to <3>,

wherein the poorly-water-soluble physiologically active compound is a kinase inhibitor, or polypeptide, or both.

<5> The nanoparticle according to any one of <1> to <4>,

wherein the additive substance is at least one selected from the group consisting of polyethylene glycol fatty acid ester, sorbitan fatty acid ester, or fatty acid.

<6> The nanoparticle according to any one of <1> to <5>,

wherein the additive substance is at least one selected from the group consisting of polyoxyl 40 stearate, polysorbate 80, or stearic acid.

<7> The nanoparticle according to any one of <1> to <6>,

wherein the poorly-water-soluble physiologically active compound is a pharmaceutical compound.

<8> A pharmaceutical composition including:

the nanoparticle according to any one of <1> to <7>.

<9> A method for producing a nanoparticle, the method including:

ejecting a solution including a poorly-water-soluble physiologically active compound from an ejection outlet including one or more pores each having an inner diameter of 1.0 mm or less into a poor solvent including an additive substance to thereby produce the nanoparticle, where the poor solvent is a poor solvent for the poorly-water-soluble physiologically active compound,

wherein the nanoparticle is the nanoparticle according to any one of <1> to <7>.

<10> The method according to <9>,

wherein the solution is ejected from the ejection outlet by applying vibrations to the solution.

<11> The method according to <9> or <10>,

wherein the solution including the poorly-water-soluble physiologically active compound is ejected from the ejection outlet into the poor solvent that flows.

<12> The method according to <11>,

wherein a speed at which the poor solvent flows is 0.3 m/s or faster.

The nanoparticle according to any one of <1> to <7>, the pharmaceutical composition according to <8>, and the method for producing nanoparticle according to any one of <9> to <12> can solve the above-described various problems existing in the art and can achieve the object of the present disclosure.

### REFERENCE SIGNS LIST

1: apparatus for producing a nanoparticle
2: solution ejecting unit
11: liquid column resonance droplet-ejecting unit
13: solution storage container
14: solution
19: ejection outlet
20: vibration generating unit
21: liquid droplets
61: poor solvent storage member
62: poor solvent

1: A nanoparticle comprising:
a poorly-water-soluble physiologically active compound; and
an additive substance,
wherein a relative span factor (R.S.F) of the nanoparticle satisfies formula:
0<(R.S.F)≤1.0,
a volume average particle diameter of the nanoparticle is 200 nm or less, and
the poorly-water-soluble physiologically active compound is covered with the additive substance.

2: The nanoparticle according to claim 1,
wherein the volume average particle diameter is 5 nm or greater but 150 nm or less.

3: The nanoparticle according to claim 1,
wherein the (R.S.F) satisfies: 0<(R.S.F)≤0.6.

4: The nanoparticle according to claim 1,
wherein the poorly-water-soluble physiologically active compound is a kinase inhibitor, or polypeptide, or both.

5: The nanoparticle according to claim 1,
wherein the additive substance is at least one selected from the group consisting of polyethylene glycol fatly acid ester; sorbitan fatty acid ester; and fatty acid.

6: The nanoparticle according to claim 1,
wherein the additive substance is at least one selected from the group consisting of polyoxyl 40 stearate, polysorbate 80, and stearic acid.

7: The nanoparticle according to claim 1,
wherein the poorly-water-soluble physiologically active compound is a pharmaceutical compound.

8: A pharmaceutical composition comprising:
the nanoparticle according to claim 1.

9: A method for producing a nanoparticle, the method comprising:
ejecting a solution including a poorly-water-soluble physiologically active compound from an ejection outlet including one or more pores each having an inner diameter of 1.0 mm or less into a poor solvent including an additive substance to thereby produce the nanoparticle, where the poor solvent is a poor solvent for the poorly-water-soluble physiologically active compound,
wherein the nanoparticle is the nanoparticle according to claim 1.

10: The method according to claim 9,
wherein the solution is ejected from the ejection outlet by applying vibrations to the solution.

11: The method according to claim 9,
wherein the solution including the poorly-water-soluble physiologically active compound is ejected from the ejection outlet into the poor solvent that flows.

12: The method according to claim 11,
wherein a speed at which the poor solvent flows is 0.3 m/s or faster.
